(19) United States
(12) Patent Application Publication
(10) Pub. No.: US 2022/0040359 A1
(43) Pub. Date: Feb. 10, 2022

(54) UV-C Room Air Sanitizer Light Box

(71) Applicant: Norman A. Shoenfeld, Cypress, TX (US)

(72) Inventor: Norman A. Shoenfeld, Cypress, TX (US)

(73) Assignee: S&S X-Ray Products, Inc.,, Houston, TX (US)

(21) Appl. No.: 16/985,431

(22) Filed: Aug. 5, 2020

(51) Int. Cl. A61L 9/20

(52) U.S. Cl. CPC A61L 9/20; A61L 2209/111; A61L 2209/15; A61L 2209/134; A61L 2209/12

(57) ABSTRACT

Wall-mounted UV-C sanitizer light box has ultraviolet lamp elements and a cover or shade movable between operating positions. In a closed position in the lamp elements are covered. In an optional intermediate position the lamp elements can emit ultraviolet to the upper part of the room space above head level. In a full open position room surfaces and furniture in the room are exposed to germicidal irradiation. The unit is fully programmable as to mode of operation and duration. Sensors can shut the unit off if persons are present or motion is detected in the room space during sanitizing. An indicator light show the status of the arrangement, i.e., whether the most recent sanitization operation was completed successfully. The shade may have built-in louvers, wherein an upper room air sanitization operation can occur with the shade in the raised or closed condition.

### BACKGROUND OF THE INVENTION

This invention relates to devices and equipment for purifying air within a space to be occupied by a person, and/or for sanitizing or sterilizing surfaces and furniture in the space that is to be occupied. The invention is more particularly concerned with sanitizing and purifying the space by use of ultraviolet radiation with a proper wavelength for killing or deactivating bacteria, mold spores, viruses, or other pathogens that may be present in the air or on the surfaces and equipment in the room. Suitable ultraviolet light for this purpose is considered to be in the C band of ultraviolet light, i.e., UV-C, nominally in a range of about 200 nm to 300 nm, with germicidal effectiveness peaking at around 260 to 270 nm. Light in this spectral range can be very effective in disinfecting room air without danger to persons in the room if it radiates only through the upper air above head level, i.e., between seven feet and ceiling level. The UV-C light tends to be absorbed by most surfaces, and so continuous use of upper air disinfecting in this fashion is unlikely to expose persons to critical levels near the maximum safe dose. The small amount or UV-C radiation that may reach below that level does not penetrate human skin beyond the outer layer of dead cells.

For whole room disinfection, some spaces such as hospital critical care and intensive care rooms, have employed ultraviolet lamps placed at an overhead location with the radiation aimed downward. This is seen to be effective for decontaminating surfaces where there may be bacterial, fungal, or viral deposits. However, these procedures can be carried out only when the room is completely unoccupied, and would not be effective for disinfecting the room air in any occupied space. Also, such devices have not been available which are programmable to conduct a room disinfection or decontamination for rooms and spaces that are not occupied on a continuous basis, such as radiology or physical therapy wards.

### OBJECTS AND SUMMARY OF THE INVENTION

Accordingly, it is an object of the present invention to provide a short-wavelength UV light box that is capable of programmed delivery of germicidal ultraviolet radiation to an entire enclosed space, such as a workshop, office room, or hospital room, by generating ultraviolet radiation for a given period of time, and sensing presence of persons or pets, and/or sensing motion, and halting the operation if a person or pet or something in motion is detected within the space.

It is another object to provide a light box that can be used for continuous generation of germicidal ultraviolet radiation within an overhead room space, i.e., limited to the air volume that is above about seven feet, for constant sanitization of room air without unduly exposing persons in the room to potentially harmful radiation.

A further object is to provide a disinfecting UV light box that can be placed into a full-room mode, an upper-air only mode, and an off (closed) condition.

According to an aspect of the present invention, the light box has a chassis with at least a first surface portion that can be mounted on a room wall surface and with a second surface that is generally vertical when the light box is hanging on or is fastened to the wall. There are two or (preferably) four lampholders (or pairs of lampholders) positioned on the chassis' second surface, and in them a respective UV-C generating lamp is positioned. The device may have a power of 35 watts to 150 watts for most applications. These lamps may be mercury-vapor tube discharge lamps, or another lighting device that is adapted for producing radiant energy in ultraviolet wavelengths that are effective for destroying or inactivating airborne and surface-borne pathogens such as bacteria, mold spores, and viruses. In front of these lamps there is a cover or shade. This is configured for removably covering all or some of the UV-C generating lamps. The shade is opaque to those wavelengths, and is pivotally mounted on the chassis at or below a lower edge of the chassis, with one or more pivot members defining a horizontal pivot axis. The pivot member(s) can be hinged, with a first hinge member attached to the chassis and a second hinge member attached to the shade.

In a preferred embodiment, the shade has a front shade portion disposed to be positioned to cover the UV-C generating lamps, and with left and right side portions. Optionally, the front shade portion may have at least one opening and louvers disposed at the at least one opening, and angled to permit the UV to go out, but block any ultravioled from reaching below the position of the light box, i.e., to keep all the light above about the seven foot lsevel.

A mechanical or electrical actuator has one portion affixed onto the chassis and has a second, moving portion connected to the shade. The actuator moves the shade between various positions. These positions can include a raised position in which the shade covers the UV-C generating lamps and blocks radiation from them, and a fully lowered position in which the shade is removed from being over the UV-C generating lamps such that radiation from the lamps can radiate at least horizontally therefrom and into the room. There can also be an intermediate position where the UV-C radiation is emitted only into the upper air part of the room (e.g., at seven feet or higher above the floor). Of course, in other embodiments, the shade could alternatively be configured so that in a lowered position it blocks the radiation from the lamps, and is raised to an intermediate position for upper air disinfection and fully raised for disinfecting the entire room.

In a preferred embodiment the shade front portion is formed as a plate member having a somewhat J-shaped profile, and the left and right side portions of the shade are each a plate member having a generally J-shaped edge. The actuator(s) favorably has a movable rod portion connected to one of the left and right side portions.

The UV room sanitizer light box can be operative to carry out a continuous upper room air sanitization procedure, limiting the ultraviolet radiant energy to room air above a predetermined head level, with the shade being positioned in its intermediate position.

The operating circuitry for energizing and controlling said UV-C generating lamps and the actuator mechanism(s) is favorably contained within said chassis enclosure, and a control panel for selecting mode of operation of the sanitizer light box device may also be positioned on the chassis.

A presence detector and/or a motion detector senses the presence of an individual within the room wherein said room sanitizer light box is mounted, or detects motion within the room and is operative to interrupt or delay energizing the UV-C generating lamps when motion or presence of an individual is detected. The operating circuitry may also include a short-range radio feature (e.g., Bluetooth or WiFi) for communicating with a smart-phone or similar hand-held device to select an operating mode or to permit remote programming of the operating system from the hand-held device.

Alternatively, the UV room sanitizer light box can have the shade or cover modified or even omitted, with the operating circuitry being configured for controllably energizing the various UV-C light generating lamp, so that lighting only some of the lamps the device carries out upper air room disinfection, and so that with all of the lamps energized the entire room is illuminated with the UV-C radiation for surface sterilization.

The operating circuitry can include a programmed functionality configured for holding the UV-C light generating lamp(s) off until a predetermined time delay after actuation of a sanitation operation (e.g., 15 seconds), then moving the shade from the closed to the fully open position, energizing at least one UV-C light generating lamp, continuing until a predetermined sanitation period has expired, and then de-energizing the UV-C light generating lamp and returning said shade to its closed position. The presence sensor and/or motion sensor act to de-energize the UV-C light generating lamp off and return the shade to its closed position when such sensor detects presence of a person during the sanitation period.

In summary, the device is in the form of a lightbox that houses two to four UV tubes/bulbs, or corresponding arrays of UV-C LEDs, when available. The configuration may vary, but the power output wattage should preferably be between about 35 and 140 W. In front of the light sources is a rotating (or lifting) cover or shade that positions in the front of the unit and provides concealment of the UV sources. The cover itself may be of a material that can be painted or may be of a material transparent to visible wavelengths but opaque to the UV-C radiation. Depending on the configuration, the cover or lid may move vertically up or down to go from a covered or closed condition to a partially open condition and a fully open condition, with a vertical lift or rotation mechanism. The device may also have a control panel that may include one or two momentary contact switches and/or a membrane touch pad or other touchpad.

For operation, the unit may be activated by pushing the momentary contact switch or the memory touch pad, or in some embodiments by actuating an app in a cell phone or other portable hand-held device. A microprocessor-controlled circuit board in the chassis dan constitute the heart of the operating circuitry.

Favorably, in a typical usage, the unit is activated, using the switch or touchpad, and then the operator leaves the room and closes the door. After a programmable time delay (e.g., fifteen seconds) the unit turns on. The cover or shade lifts or rotates away to expose the UV-C tubes or bulbs. The lamps remain on for the programmed time period for a disinfection cycle (e.g., between fifteen minutes and one hour or in some environments, two hours). The time the lamps remain ON is fully programmable.

The unit can be set to be off and closed until activated, and then on and fully opened after a short delay (15 seconds), then at the end of the programmed sanitation cycle period, the lamps are shut off and the shade or cover is closed. During a sanitation cycle, any motion detected in the room would turn off the light, and (preferably) send the cover or shade to the closed position.

As an alternative, the unit may be configured to remain ON until the unit is shut off (or until someone walks into the room or opens the door).

The unit can be set into a continuous or intermittent upper air sanitation mode, with the shade partially open and serving as a louver, so that all the radiation is at or above the seven foot level. The unit can also be programmed normally to be in the upper room air sanitation mode, but when activated to into the fully-opened full room sanitation mode (after 15 seconds) and remain until the programmed interval (15 minutes to two hours) and then return to the partially open mode or upper room air mode. If human presence or motion is detected during the programmed interval, the unit would return to the partially open mode.

These and other configurations, features, and advantages of this invention will become apparent from the ensuing description of selected preferred embodiments of this invention, with reference to the accompanying Drawing.

### BRIEF DESCRIPTION OF THE DRAWING

FIG. 1 is a front elevational view of a room air sanitizer light box according to one embodiment of this invention.

FIG. 2 is a side elevation cut away at line 2-2 of FIG. 1 for explaining this embodiment.

FIG. 3 is a front elevation thereof showing the shade or cover thereof lowered or rotated down to a full open position.

FIG. 4 is a side elevation cut away at line 4-4 of FIG. 3 showing the shade or cover in the full open position.

FIG. 5 is a perspective view thereof from above showing the left portion of the room air sanitizer light box, the corresponding right portion thereof being substantially a mirror image.

FIG. 6 is a schematic side view of the shade or cover and corresponding actuator for illustrating the closed, fully open, and partially open (i.e., upper room air sanitation) positions of the shade or cover.

FIG. 7 is a perspective environmental view showing the room air sanitizer light box of this embodiment as typically mounted on a wall of a room.

FIG. 8 is a diagram explaining the functionalities of the room air sanitizer of this invention.

FIGS. 9 and 10 are side views showing the room air sanitizer light box of another preferred embodiment of the invention, in raised (closed) and lowered (open) positions, respectively.

### DETAILED DESCRIPTION OF THE PREFERRED EMBODIMENTS

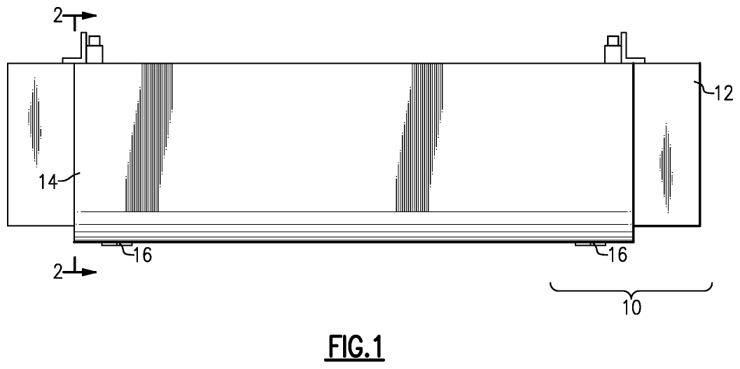

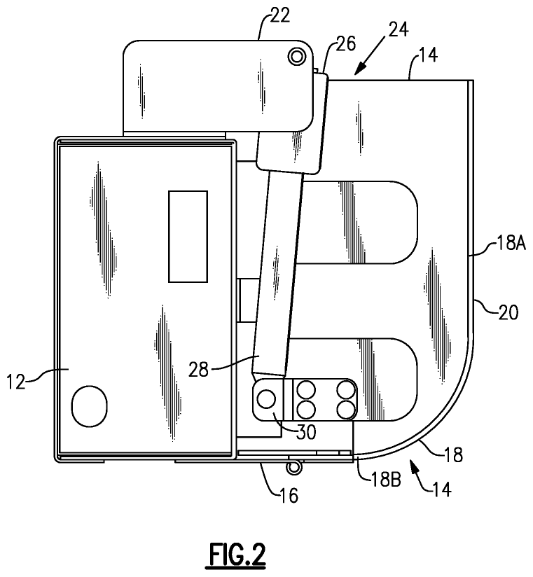

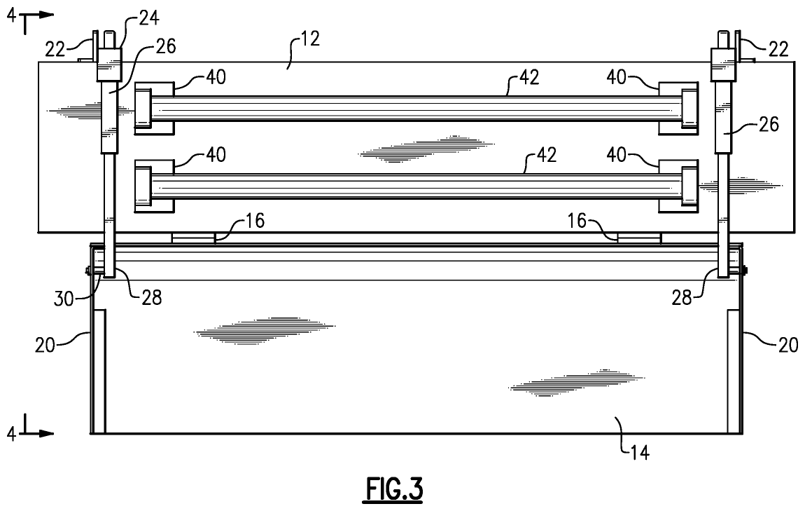

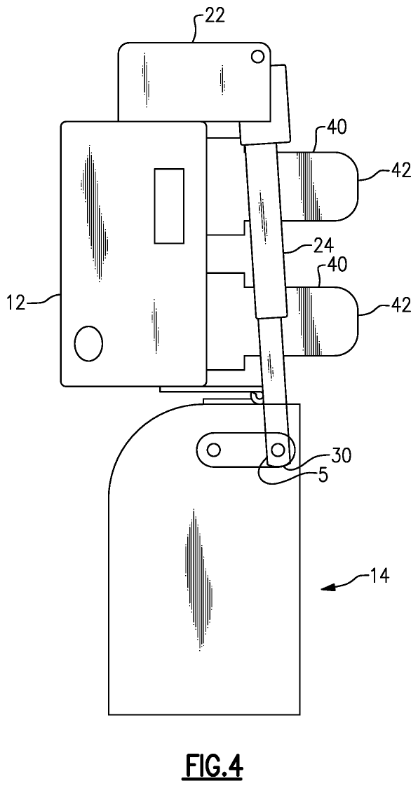

With reference to the Drawing, and initially to FIGS. 1 and 2 thereof, a wall-mounted room air sanitizer, in the form of a UV-C light box assembly 10, is shown as having a main chassis 12 which contains electrical circuitry and controls as well as ultraviolet-emitting equipment. A cover or shade 14 is joined by one or more hinges 16 to the lower side of the chassis 12, so that shade 14 is configured to swing up to its closed position as shown in FIGS. 1 and 2, and swing down about 180 degrees to its full open position, as illustrated in FIGS. 3 and 4. The cover 14 is formed of a curved front plate 18, here spanning most of the width of the chassis except for the right and left ends, and of similar height to the chassis. The front plate 18 has a generally flat front portion 18A, and a curved lower portion 18B, so that the shade has a generally J-shaped profile. There are left and right sidewalls 20, 20 which here are generally flat, and with a profile that matches that of the front plate 18, i.e., generally rectangular, but with a curved lower front corner.

Here the chassis 12 here is in the form of a box or enclosure which contains electrical and control circuitry (not shown) as needed for powering and controlling ultraviolet lamps and raising and lowering the cover or shade 14 as will be described presently. The rear wall of the chassis 12 is adapted for mounting on a wall of the room, suitably at an elevated position (e.g., seven feet or about 215 cm) above the floor. The unit may be corded for plugging into an existing wall outlet, or may be wired directly to a dedicated circuit.

As shown in the side elevations of FIGS. 2 and 4, the hinges 16 provide a rotation axis that is at or near the base side of the chassis 12, and slightly forward (distal) of the front wall of the chassis. Pivot mounting plates 22 are affixed to the top of the chassis 12 near respective right and left ends of the shade 14.

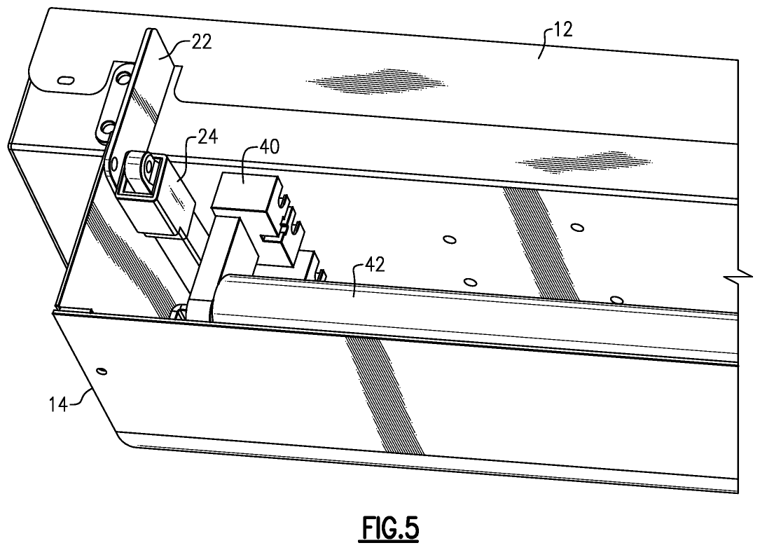

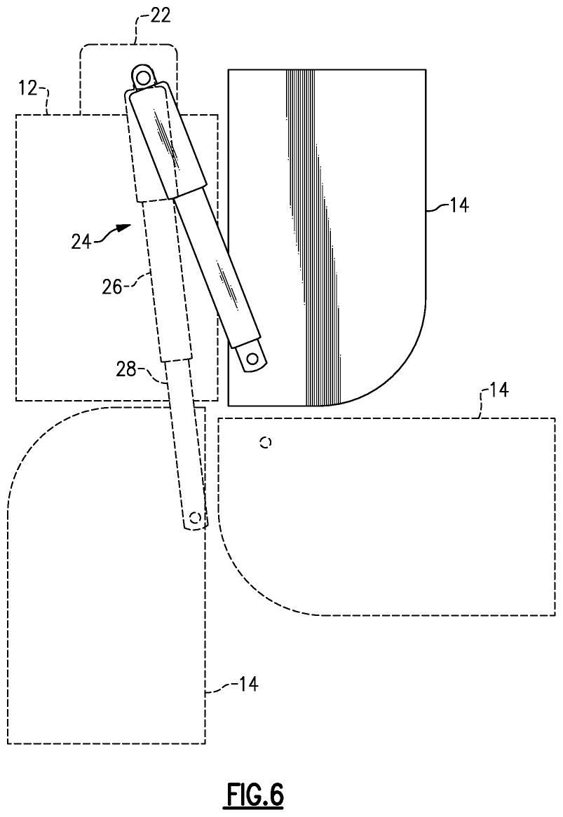

One or more linear actuators 24, here a pair of actuators, are provided, in this embodiment with a main cylinder body 26 pivotally attached to the respective one of the mounting plates 22, and a rod or lead screw 28 connected to a respective mounting bracket 30 situated on the respective right or left wall 20 of the shade. The actuators are controlled so as to move the cover or shade 14 between the raised or closed position (FIGS. 1, 2 and in the perspective view of FIG. 5) and the full open position (FIGS. 3, 4) and to at least one intermediate position, as discussed later in connection with upper room air sanitization. The various closed, full open, and partially open positions of the shade 14 relative to the chassis 12 are illustrated schematically in FIG. 6.

As shown in FIGS. 3, 4, and 5 there are lamp holders 40 affixed onto the front side of the chassis, and these are configured to hold ultraviolet lamps, here shown as discharge tubes capable of emitting ultraviolet radiation in the C band or very short wavelength band between about 200 nm and 280 nm, and favorably at about 250 to 260 nm. These wavelengths have excellent germicidal effects for killing bacteria and mold spores and inactivating viruses. On the other hand, these wavelengths are strongly absorbed by surfaces in the room and are considered safe so long as the UV-C light does not fall directly on a person. At present, mercury vapor discharge lamps are used for generating ultraviolet light in the C band. Other luminaires could be used, and semi-conductor lasers and LED's emitting UV-C either are available or are in development and are expected to be available in the near future.

As discussed earlier, the light box unit 10 can favorably be mounted at or above the head level of persons that might be standing in the room, so that when the shade 14 is in its intermediate or partly open position, the ultraviolet illumination is limited to the upper room air zone, i.e., between seven feet and the ceiling. In that mode, the light box assembly can be left on continuously when the room is occupied, for purposes of destroying pathogens that may be present in the room air. For purposes of sanitizing surfaces in the room, the shade 14 is lowered and fully opened so that the UV-C radiant energy can reach down to walls, tables, and furniture to destroy accumulations of virus and bacteria in the room. In the OFF mode, the cover or shade is normally raised to its closed position.

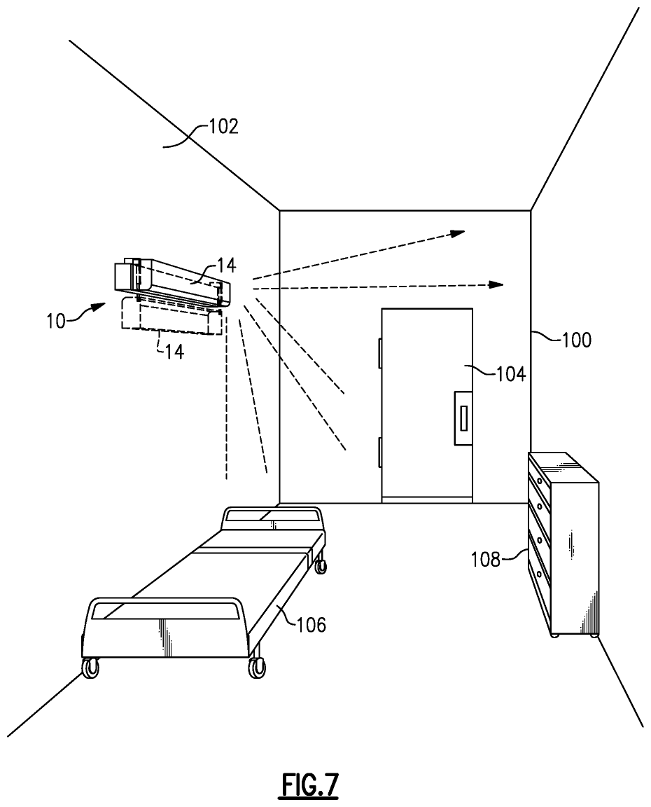

FIG. 7 shows an example of a room where this unit may be employed, e.g., a treatment room or therapy room 100, with the UV-C light box assembly 10 mounted on an upper part of one room wall 102. When the room is occupied, i.e., if a patient and therapist are together in the room, the light box assembly would either be OFF or set in the upper air sanitation mode in which the shade 14 would be lowered partway to an intermediate position. In the latter mode, any UV radiation would reach only portions of the room above head level of the occupant(s). At given pre-programmed times, or when commanded (e.g., by pushing a control) the light box assembly goes into a full room sanitation mode, in which the shade 14 is fully lowered so that the sterilizing radiation can reach surfaces of furniture and equipment throughout the room, such as the room door 104, a patient bed 106 or cabinet 108, as well as wall and floor surfaces that are not in shadow. The full room sanitation mode can be commenced by pressing a button on a control panel, or from a hand-held device connected by wireless or Bluetooth or infrared to the light box unit 10. Then there is a short delay period, between fifteen seconds and a minute, to allow personnel to exit the room and close the door 104. Then, the light box opens and energizes the UV lamps 42, and continues for a programmed length of time, between a few minutes and an hour typically depending on conditions and need for sanitation. At the end the lamps are shut off and the cover or shade 14 is returned to its closed position. Alternately, at the end of the full room sanitation, the lamps 42 can remain on that the shade moved to the partial open position for upper room air germicidal cleansing. The light box assembly 10 is favorably provide with presence detection and motion detection functionalities, so that if person is detected in the room, or if there is motion detected, such as the door 104 being opened, the light box will immediately go to its OFF mode (or alternatively to its upper room air sanitation mode).

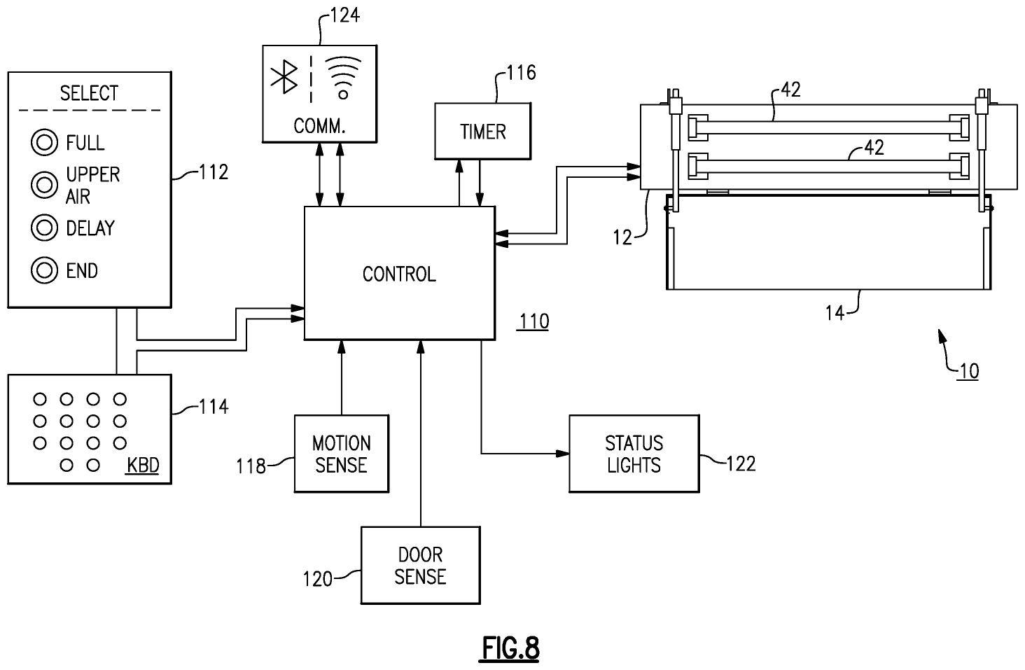

FIG. 8 is a schematic illustration for explaining the functionality and operation of the germicidal and anti-viral UV-C sanitizer light box assembly 10. A main control circuit 110 (here shown only diagrammatically) is a part of the electrical circuitry within the sanitizer chassis 12, and controls its operation. This may be based on a microprocessor that can be programmed. This is connected with the lampholders 40 and actuators 24 to control the opening and closing of the shade 14 and the lighting of the UV-C lamps 42. A keypad, e.g., a membrane touchpad, may be positioned on one of the end panels of the chassis 12, or at a convenient place on the room wall 112. This may have a number of membrane switch buttons permitting a user to select and start a given mode of operation, i.e., full room sterilization or upper air germicidal operation, or to insert a delay before a given operation is commenced, or to halt or end an operation. This may also include a numeric or alphanumeric keypad 114, which may permit altering the program on the microprocessor, i.e., to change the cycle time for a sterilization within a range of perhaps fifteen minutes to two hours. A timer functionality 116 serves to allow the control circuit 110 to turn the unit on or off automatically or to go from one mode to another at a given programmed time. A motion detector 118 and/or a presence sensor 120 detect if there is motion in the room or it a person is present. These allow the sanitizer light box assembly to shut off automatically from a full room sanitization procedure if a person is detected in the space, or if motion is sensed, such as the door 104 being opened. These can employ a passive infrared sensor or a microwave radar sensor. The control circuit 110 can also control a status light or lights 122, e.g., color LEDs that glow red or green for example to identify whether the last sterilization was completed successfully or was interrupted; whether the operation has been completed within the previous twenty-two hours or if it is time for another sterilization operation, There can also be a status light that blinks an error code if a misoperation is detected. The control circuit 110 may also be provided with a wireless communication feature 124, which may include one or more of local wireless (Wi-Fi), or Bluetooth, or an infrared wireless connection. The light box assembly 10 may also be wired to a network, e.g., by Ethernet cable. A further possibility is to provide a wireless connection to a smart phone or other hand-held device

Preferably, the lightbox assembly is fully programmable. The unit can be set to be OFF/closed until activated, and then ON and fully opened after e.g. 15 seconds for the programmed time interval, and then OFF with the cover or shade closed. In this mode, if motion is detected or if the door is opened, the illumination would end and the cover or shade would return to the closed position. This is considered normal operating mode. Alternatively, the unit can be programmed to remain ON after being actuated until it is turned off or until someone enters the room or opens the door. Another mode is that the unit will be operated for upper room air germicidal sanitation as the normal mode, and when activated, after the initial 15 second delay, would to into the full room sterilization mode, with the lid fully opened, and then when the full room sterilization period ends (or motion is detected) the unit returns to the upper room air mode with the shade 14 partially open. In yet another mode, the light box assembly 10 can be set to go into the upper room air mode (with the shade partially open) and after a programmed time of minutes or hours, return to the OFF mode with the shade raised and closed. The unit may also be programmed, when the unit is in the upper air sterilization mode to detect if there is motion above the nominal seven foot level (e.g. if someone is working on a ladder in the room), and automatically interrupt operation. In the case of an interrupted full room sanitization operation (due to motion or human presence being detected) the system can be programmed either to automatically restart after a suitable delay time when the motion or presence is no longer detected, or to cancel the until manually restarted or until the next programmed operation.

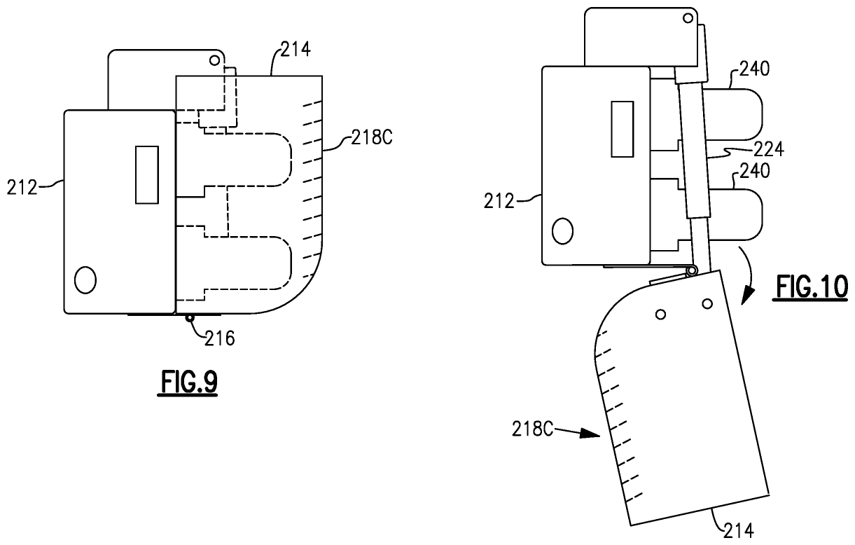

FIGS. 9 and 10 show a further embodiment of the UV-C light box assembly 210 of this invention, in which louvers are incorporated into the cover or shade thereof so that upper room air sanitation can take place without having to lower or rotate the shade. Elements similar to those of the first embodiment are identified with the same reference numbers, but raised by 200. Here the light box assembly 210 has a chassis 212 and cover or shade 214 joined by hinges 216, as in the first-described embodiment, with lampholders 240 and UV-C emitting lamps (obscured in these views), which are concealed behind the shade 214 in the normal or raised position of FIG. 9. However, rather than a solid unperforated front plate as in the prior embodiment, the front surface of the shade 214 has a series of upwardly slanted louvers 214C formed in the front of the plate ahead of the lamps with horizontal slot openings between the louvers. In this embodiment, the louvers are disposed on the interior of the shade. The louvers 218C allow radiation from the lamps to be emitted forward and upward, but prevent the radiation from extending downward below the light box, i.e., below the seven-foot level. The interior louvers 218C allow the shade 214 to be fully lowered without it hitting onto the wall on which the assembly 210 is mounted. With this assembly 210, the upper room air sanitation mode is carried out with the shade 214 in the raised position (FIG. 9), and the actuator or actuators (224) lower the shade 214 to the full open position (FIG. 10) for the full room sanitizing mode. There is no need for the intermediate, partly open position for the shade.

In a still further embodiment of this invention, the shade 314 can include an extension plate that carries a number of louvers or fins. For the upper room air sanitizing mode of operation, the actuator(s) lower the shade to its intermediate or partly open position which places the louvers in front of the lampholders and their UV-C lamps, so that ultraviolet radiation is directed at an upward angle and does not go below about the seven foot level. The shade or cover can be lowered to the fully open position for full room germicidal sanitation. The series of louvers can be coupled by suitable linkage so that in the closed or raised position, the stack of louvers can rotate or slide to a more forward position and will not collide with the wall on which the assembly is mounted.

As an alternative to the touchpad mentioned earlier, the unit may have two or more momentary contact switches for initiation of a sterilization sequence and for programming the unit, e.g., as regards length of time the UV-C illumination is to last.

Preferably, in each of these embodiments there will be an indicator red/green LED pilot light or indicator, and two momentary contact switch buttons for controlling and programming the light box. The LED(s) will show the program/operating mode, which indicate the action according to the following table:

| Button 1 | Button 2 | Action | LED response |
|---|---|---|---|
| ON | OFF | Start 15 second | RED alternating |
| | | counter for room | on/off |
| | | sterilization | |
| ON (hold 10 | OFF | Enter program mode | GREEN alternating |
| seconds) | | | on/off |
| ON (hold 5 | OFF | Exit program mode | 1 RED flash, 4 |
| seconds) | | | GREEN flashes |

While in Program Mode: (Button 2 not used until EXIT—see above)

| Button 2 | Action | LED (GREEN) |
|---|---|---|
| ON (once) | Standard (default) operation | 1 flash |
| ON (two times) | Always on upper air sterilization | 2 flashes |
| ON (three times) | 60 minute sterilization | 3 flashes |
| ON (four times) | 90 minute sterilization | 4 flashes |
| ON (five times) | 120 minute sterilization | 5 flashes |
| ON (six times) | 160 minute sterilization | 6 flashes |
| ON (seven times) | 240 minute sterilization | 7 flashes |

The aforementioned membrane touchpad switch may alternatively be used for programming, and may be required for security, namely, for entering a passcode to commence a start sequence after delay, or to enter programming mode. The red LED indicator may also be configured to flash an error code if there is trouble, which may be useful in troubleshooting.

In place of the linear actuator(s) described here, a servomotor may be used to rotate the cover or shade between positions. Also, additional vanes or louvers may be used when needed to confine the sterilizing radiation to the upper portion of the room. In some possible designs, the shade may be raised rather than lowered to move from the closed to the open position. In some alternatives to the above-described embodiment, the chassis and lamps may lift (or rotate) relative to a shade that is fixed in place on a wall or other fixed support. In this embodiment two UV-C tubes or lamps are employed but in many applications four or more lamps may provide superior performance. Other possible embodiments could employ individual shades or louvers for the respective tubes or bulbs, and/or control the ON/OFF state of the respective tubes or bulbs individually to control the direction of illumination.

While the invention has been described in terms of selected preferred embodiments, it should be understood that the invention is not limited only to those embodiments, but rather the scope of this invention is to be measured by the appended claims.

What is claimed is:

1. UV room sanitizer light box device comprising
a chassis having a first surface for mounting on a room wall surface and a second surface having a lower edge;
a plurality of lampholders positioned on said second surface, and a plurality of UV-C generating lamps mounted in said lampholders, and being adapted for producing radiant energy in ultraviolet wavelengths effective for destroying or inactivating airborne and surface-borne pathogens;
a shade configured to removably cover said UV-C generating lamps, and pivotally mounted on said chassis at or below a lower edge of said second surface, including at least one pivot member having a horizontal pivot axis and having a first member attached to the chassis and a second member attached to the shade, said shade having a shade front portion disposed to be positioned to cover said UV-C generating lamps and left and right side portions;
at least one actuator affixed onto said chassis and having a moving portion connected to said shade and operative to move said shade between a raised position in which said shade is over said UV-C generating lamps and blocks radiation therefrom, and a fully lowered position in which the shade is removed from being over the UV-C generating lamps such that radiation from said lamps can radiate at least horizontally therefrom and into said room.

2. The UV room sanitizer light box device according to claim 1, wherein said shade front portion is formed as a plate member having a J-shaped profile.

3. The UV room sanitizer light box device according to claim 2, wherein said left and right side portions of said shade are each a plate member having a generally J-shaped edge.

4. The UV room sanitizer light box device according to claim 1, wherein said at least one actuator has a movable rod portion connected to one of said left and right side portions.

5. The UV room sanitizer light box device according to claim 1, wherein said at least one actuator is further operative to move said shade to an intermediate position between said raised position and said fully lowered position.

6. The UV room sanitizer light box device according to claim 5, wherein said sanitizer light box device is configured to carry out a continuous upper room air sanitization procedure, limiting the ultraviolet radiant energy to room air above a predetermined head level, and including placing said shade in said intermediate position and energizing said UV-C generating lamps.

7. The UV room sanitizer light box device according to claim 1, wherein said chassis is formed as an enclosure disposed between said first surface and said second surface thereof, with operating circuitry for energizing and controlling said UV-C generating lamps being contained within said enclosure.

8. The UV room sanitizer light box device according to claim 7, further comprising a control panel positioned on said chassis for selecting mode of operation of said sanitizer light box device.

9. The UV room sanitizer light box device according to claim 7, further comprising a presence detector for detecting the presence of an individual within the room wherein said room sanitizer light box is mounted, and said operating circuitry is operative to interrupt or delay energization of said UV-C generating lamps when presence of an individual is detected.

10. The UV room sanitizer light box device according to claim 7, wherein said operating circuitry includes a short-range radio mechanism for communicating with a hand-held device to permit remote programming of said operating system from said hand-held device.

11. UV room sanitizer light box device of the type comprising a chassis mountable upon a wall of a room, and which has thereon at least one UV-C light generating lamp configured to produce radiant energy in ultraviolet wavelengths effective for destroying or inactivating airborne and surface-borne pathogens; a shade movably positioned relative to said chassis to obstruct at least a portion of the light emitted from said at least one UV-C light generating lamp; operating circuitry disposed within said chassis for energizing and controlling said at least one UV-C generating lamp, the operating circuitry being configured for controllably adjusting the relative position of said shade and said chassis between a closed position in which said shade blocks the radiant energy of said at least one UV-C light generating lamp; a fully open position in which the radiant energy radiates to upper and lower portions of said room; and an intermediate position between said closed and fully open positions in which the radiant energy radiates only to an upper portion of the room.

12. UV room sanitizer light box device according to claim 11 further comprising at least one linear actuator coupled between said light box chassis and said shade for adjusting the relative position of said chassis and said shade between said closed, intermediate, and fully open positions.

13. UV room sanitizer light box device according to claim 12 in which said linear actuator is operative to rotate said shade relative to said chassis between said closed, intermediate, and fully open positions.

14. UV room sanitizer light box device according to claim 11 wherein said operating circuitry includes a programmed functionality that is configured to hold the at least one UV-C light generating lamp off until a predetermined time delay after actuation of a sanitation operation, then adjusting the relative position of the chassis and the shade from the closed to the fully open position, energizing said at least one UV-C light generating lamp, continuing until a predetermined sanitation period has expired, and then de-energizing said at least one UV-C light generating lamp and returning said chassis and said shade to the closed position.

15. UV room sanitizer light box device according to claim 14 further comprising a presence sensor mounted on or in said chassis, and wherein said operating circuitry is configured to energize said UV-C light generating lamp off and return said shade to its closed position when said sensor detects presence of a person during said sanitization period.

16. UV room sanitizer light box device according to claim 11 wherein said operating circuitry includes a programmed functionality that is configured to energize the at least one UV-C light generating lamp on in normal operations, and to maintain the shade and the chassis in the intermediate position, and upon activation of a sanitation operation, after a predetermined time delay after said actuation, then adjusting the relative position of the chassis and the shade from the intermediate position to the fully open position, continuing until a predetermined sanitation period has expired, and returning the chassis and shade to the intermediate position.

17. UV room sanitizer light box device according to claim 11 comprising at least oe indicator light and wherein said operating circuitry is configured to detect whether a full room sterilization operation has been initiated and whether such operation has been successfully completed; and said operating circuit is operative to illuminate said at least oe indicator light according to a predetermined pattern when said full room sterilization operation has been successfully completed, and to illuminate said at least one indicator light according to a different predetermined pattern when said sterilization operation has terminated before completion.

18. UV room sanitizer light box device according to claim 17, wherein said at least one indicator light is operative to provide visual confirmation of the programming mode of the operating circuitry.

19. UV room sanitizer light box device of the type comprising a chassis mountable upon a wall of a room, and which has thereon a plurality of UV-C light generating lamps configured to produce radiant energy in ultraviolet wavelengths effective for destroying or inactivating airborne and surface-borne pathogens; and operating circuitry disposed within said chassis for energizing and controlling said at least one UV-C generating lamp, the operating circuitry being configured for controllably directing said radiant energy such that in a closed condition the radiant energy does not exit the light box into the room; a fully open condition in which the radiant energy radiates to upper and lower portions of said room; and an intermediate condition which the radiant energy radiates only to the upper portion of the room.

20. The UV room sanitizer light box device according to claim 19, wherein said operating circuitry includes a short-range radio mechanism for communicating with a hand-held device to permit remote programming of said operating system from said hand-held device.

21. UV room sanitizer light box device of the type comprising a chassis mountable upon a wall of a room, and which has thereon at least one UV-C light generating lamp configured to produce radiant energy in ultraviolet wavelengths effective for destroying or inactivating airborne and surface-borne pathogens; a shade movably positioned relative to said chassis to obstruct at least a portion of the light emitted from said at least one UV-C light generating lamp; operating circuitry disposed within said chassis for energizing and controlling said at least one UV-C generating lamp, the operating circuitry being configured for controllably adjusting the relative position of said shade and said chassis between a closed position in which said shade at least partially blocks the radiant energy of said at least one UV-C light generating lamp; and a fully open position in which the radiant energy radiates to upper and lower portions of said room; and wherein said shade has at least one opening in a front thereof and a plurality of louvers aligned with said at least one opening and positioned such that light from said at least on UV-C light generating lamp can exit said at least one opening between said louvers, but is prevented from radiating downward into the room below said light box device.

22. The UV room sanitizer light box device according to claim 21, wherein said louvers project inward from the at least one opening towards said at least one UV-C light generating lamp.
